(19) United States
(12) Patent Application Publication
(10) Pub. No.: US 2012/0022099 A1
(43) Pub. Date: Jan. 26, 2012

(54) NOVEL POLYMORPHIC FORMS OF AN AZABICYCLO-TRIFLUOROMETHYL BENZAMIDE DERIVATIVE

(75) Inventors: Evgeny ZLOTNIKOV, Highland Park, NJ (US); Xiao-Dong WU, Bridgewater, NJ (US); Harvey LIEBERMAN, Hoboken, NJ (US); Boris GORDONOV, East Windsor, NJ (US); Timothy DONEGAN, Philadelphia, PA (US); Diana SHADEED, Easton, PA (US); Yushen GUO, Basking Ridge, NJ (US)

(73) Assignee: SANOFI, Paris (FR)

(21) Appl. No.: 13/151,914

(22) Filed: Jun. 2, 2011

Related U.S. Application Data
(63) Continuation of application No. PCT/US2009/066515, filed on Dec. 3, 2009.
(60) Provisional application No. 61/119,811, filed on Dec. 4, 2008.

(51) Int. Cl. A61K 31/439; A61P 25/24; A61P 25/18; A61P 25/22; C07D 453/02; A61P 25/00

(52) U.S. Cl. 514/305; 546/133

(57) ABSTRACT

The present disclosure is directed to solid forms of the compound of formula (I):

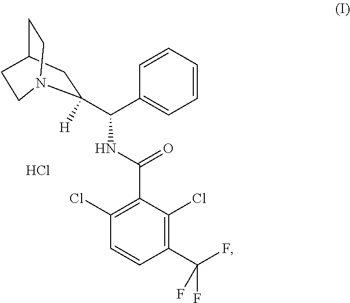

to compositions comprising these forms, and to processes for their preparation. The disclosure also relates to methods for the treatment of neurological disorders through the administration of these forms.

This application is a continuation of International Application No. PCT/US2009/066515, filed Dec. 3, 2009, which claims the benefit of priority of U.S. Provisional Application No. 61/119,811, filed Dec. 4, 2008, both of which are incorporated herein by reference.

### FIELD OF THE INVENTION

The present invention relates to novel forms of N—[(S)-2(S)-1-azabicyclo[2.2.2]oct-2-yl(phenyl)methyl]-2,6-dichloro-3-(trifluoromethyl)benzamide hydrochloride and pharmaceutical compositions thereof. This invention also relates to processes for the preparation of such forms and pharmaceutical compositions, and to methods of use thereof for the treatment of disorders related to GLYT-1.

### BACKGROUND OF THE INVENTION

Current anti-psychotic drugs are only partially effective in treating schizophrenia and there is a clear need to develop better drugs for the therapeutic treatment thereof. Traditional models of schizophrenia have focused primarily upon dopaminergic dysregulation. In contrast, more recent models focus on dysfunction of glutamatergic systems, acting particularly through N-methyl-D-aspartate (NMDA) receptors. More specifically, stimulation of NMDA receptors with glycine site agonists may have therapeutic effects and a number of clinical trials of glycine together with standard anti-psychotic drugs have recently been conducted. Modest improvements in negative symptoms have been reported in some studies but a potentially more effective treatment is to use inhibitors of the GLYT-1 subtype of glycine transporters. Expression of GLYT-1 within the brain correlates with NMDA receptor expression patterns, and it has been suggested that GLYT-1 may regulate synaptic glycine concentrations. With the development of selective and potent non-transported inhibitors of GLYT-1, it should be possible to elevate synaptic glycine concentrations more effectively and thereby to increase NMDA receptor activity. Recent in vitro studies demonstrate that the glycine transport inhibitor, N-[3-(4-fluorophenyl)-3-(4′-phenylphenoxy)]propylsarcosine, enhances NMDA receptor activity and the use of this class of compounds in clinical studies is eagerly awaited.

NMDA receptors in the brain are regulated by glycine, acting via a strychnine-insensitive regulatory site, and by GLYT-1 that maintain low glycine levels in the immediate vicinity of the NMDA receptor complex. It is known that NMDA receptors are involved in the modulation of striatal dopamine release in vitro, and may interact with glycine transport inhibitors (GTIs) as potential psychotherapeutic agents in schizophrenia. In the striatum, NMDA receptors exert dual excitatory/inhibitory effects, with inhibition reflecting activity of local GABAergic feedback regulation.

The effectiveness of glycine in regulating [³H]DA release both in vivo and in vitro is consistent with its beneficial clinical effects. Similar effects have been shown for the high-affinity GTI (+)N-[3-(4′-fluorophenyl)-3-(4′-phenylphenoxy)-propyl]sarcosine (NFPS), and for a range of high-affinity GTIs with appropriate rank order of potency. A known potent and selective GLYT-1 inhibitor (N-[3-(4′-fluorophenyl)-3-(4′-phenylphenoxy)propyl]sarcosine [NFPS]) provides a tool that suggests that inhibition of GLYT-1 may increase synaptic glycine and thereby potentiate NMDA receptor function in vivo. In addition, (+)-NFPS significantly stimulated NMDA-induced [³H]GABA release. It has been shown that (+)-NFPS demonstrates a greater than 10-fold activity in an in-vitro functional glycine reuptake assay relative to the racemic compound. In vivo, (+/−)-NFPS significantly enhanced long-term potentiation in the hippocampal dentate gyrus induced by high-frequency electrical stimulation of the afferent perforant pathway. Furthermore, (+)-NFPS induced a pattern of c-Fos immunoreactivity comparable with the atypical anti-psychotic clozapine and enhanced pre-pulse inhibition of the acoustic startle response in DBA/2J mice, a strain with low basal levels of pre-pulse inhibition. This suggests that selective inhibition of GLYT can enhance NMDAR-sensitive activity in vivo and also support the idea that GLYT-1 may represent a novel target for developing therapeutics to treat disorders associated with NMDA receptor hypofunction. The effects were blocked by the glycine-site antagonists L689,560 and HA-966, and the GABA_{B} antagonists phaclofen and CGP 52432, confirming the roles of both the NMDA-associated glycine-site and pre-synaptic GABA_{B} receptors in NMDA receptor-mediated regulation of striatal D-aspartate release in vitro.

Endogenous D-aspartate hyper-activity is associated with prominent positive symptoms in schizophrenia and glycine-site agonists and GTIs have been shown to be effective in the reduction of persistent positive, as well as negative, symptoms in schizophrenia. Glycine acts as a necessary co-agonist for glutamate at the N-methyl-D-aspartate NMDA receptor (NMDA) complex by binding to the strychnine-insensitive glycine-B binding site on the NR1 subunit. The fact that glycine is normally found in the brain and spinal cord at concentrations that exceed those required to saturate this site has led to the speculation that glycine normally saturates NMDA receptor-containing synapses in vivo. However, additional lines of evidence suggest that synaptic glycine may be efficiently regulated in synaptic areas by the glycine transporter type 1 (GLYT-1).

Hypofunction of the N-methyl-D-aspartate glutamate receptor has been implicated in the pathophysiology of schizophrenia. Treatment with D-serine or glycine, endogenous full agonists of the glycine site of N-methyl-D-aspartate receptor, or D-cycloserine, a partial agonist, have been shown to improve the symptoms of schizophrenia. N-methylglycine (sarcosine) is an endogenous antagonist of glycine transporter-1, which potentiates glycine's action on N-methyl-D-aspartate glycine site and has been shown to also have beneficial effects on schizophrenia and its symptoms.

N—[(S)-2(S)-azabicyclo[2.2.2]oct-2-yl(phenyl)methyl]-2,6-dichloro-3-trifluoromethyl)benzamide hydrochloride, which has the structure of formula (I):

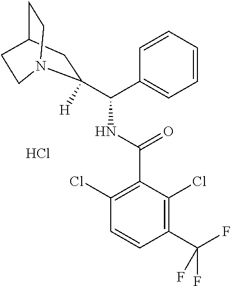

is a specific inhibitor of the glycine transporter GLYT-1. The preparation, physical properties, and beneficial pharmacological properties of N—[(S)-2(S)-azabicyclo[2.2.2]oct-2-yl(phenyl)methyl]-2,6-dichloro-3-trifluoromethyl)benzamide hydrochloride are described in, for example, U.S. Pat. No. 7,288,656 (also WO2005/037783).

The large-scale manufacturing of a pharmaceutical composition poses many challenges to the chemist and chemical engineer. While many of these challenges relate to the handling of large quantities of reagents and control of large-scale reactions, the handling of the final product poses special challenges linked to the nature of the final active product itself. Not only must the product be prepared in high yield, stable, substantially free of impurities and capable of ready isolation, the product must possess properties that are suitable for the types of pharmaceutical preparations in which they are likely to be ultimately used. The stability of the active ingredient of the pharmaceutical preparation must be considered during each step of the manufacturing process, including the synthesis, isolation, bulk storage, pharmaceutical formulation and long-term formulation. Each of these steps may be impacted by various environmental conditions of temperature and humidity.

The pharmaceutically active substance used to prepare the pharmaceutical compositions should be as pure as possible and should have long term stability under various environmental conditions. This is important to prevent the appearance of unintended degradation products in pharmaceutical compositions, which degradation products may be potentially toxic or result simply in reducing the potency of the composition.

The preparation and isolation of the stable polymorphic form of N—[(S)-2(S)-1-azabicyclo[2.2.2]oct-2-yl(phenyl)methyl]-2,6-dichloro-3-trifluoromethyl)benzamide hydrochloride, Form A, from selected organic solvents resulted in the production of drug substance with unacceptable levels of residual solvents. In order to remove the solvents entrapped by the crystals, the substance needs to be dried through heating of up to about 220° C. However, release of residual solvents at temperatures above 220° C. is also accompanied by thermal decomposition of the compound.

Therefore, methods for preparing the highly stable polymorph of N—[(S)-2(S)-1-azabicyclo[2.2.2]oct-2-yl(phenyl)methyl]-2,6-dichloro-3-trifluoromethyl)benzamide hydrochloride, Form A, in substantially pure form are clearly needed.

### SUMMARY OF THE INVENTION

Accordingly, the present invention relates to processes for preparing Form A in substantially pure form.

The present invention is also directed to novel polymorphic forms of N—[(S)-2(S)-azabicyclo[2.2.2]oct-2-yl(phenyl)methyl]-2,6-dichloro-3-trifluoromethyl)benzamide hydrochloride.

One aspect of the invention is the novel polymorphic form of N—[(S)-2(S)-azabicyclo[2.2.2]oct-2-yl(phenyl)methyl]-2,6-dichloro-3-trifluoromethyl)benzamide hydrochloride designated as Form B.

Another aspect of the invention is the novel ethanol solvate form of N—[(S)-2(S)-azabicyclo[2.2.2]oct-2-yl(phenyl)methyl]-2,6-dichloro-3-trifluoromethyl)benzamide hydrochloride.

Another aspect of the invention is the novel 2-propanol solvate form of N—[(S)-2(S)-azabicyclo[2.2.2]oct-2-yl(phenyl)methyl]-2,6-dichloro-3-trifluoromethyl)benzamide hydrochloride.

Another aspect of the invention is a polymorphic form of N—[(S)-2(S)-azabicyclo[2.2.2]oct-2-yl(phenyl)methyl]-2,6-dichloro-3-trifluoromethyl)benzamide hydrochloride designated as Form A which is substantially pure.

Another aspect of the invention is an amorphous form of N—[(S)-2(S)-azabicyclo[2.2.2]oct-2-yl(phenyl)methyl]-2,6-dichloro-3-trifluoromethyl)benzamide hydrochloride.

Another aspect of the invention is a process for preparing a pharmaceutical composition of N—[(S)-2(S)-azabicyclo[2.2.2]oct-2-yl(phenyl)methyl]-2,6-dichloro-3-trifluoromethyl)benzamide comprising formulating one or more compounds of the invention with one or more pharmaceutically acceptable carrier agents, bulking agents, solvents, diluents and other excipients

Another aspect of the present invention is a method of treating a pathology in which an inhibitor of GLYT-1 provides a therapeutic benefit.

The present invention is more fully discussed with the aid of the following figures and detailed description below.

### BRIEF DESCRIPTION OF THE DRAWINGS

FIG. 1 is an X-Ray Powder Diffraction Pattern of Form A in substantially pure form

FIG. 2 is a Differential Scanning calorimetry Trace of Form A in substantially pure form

FIG. 3 is a Fourier Transform Infrared Spectrum of Form A in substantially pure form

FIG. 4 is an X-Ray Powder Diffraction Pattern of Form B

FIG. 5 is a Differential Scanning calorimetry Trace of Form B

FIG. 6 is an Infrared Spectrum of Form B

FIG. 7 is an X-Ray Powder Diffraction Pattern of the ethanol solvate

FIG. 8 is an X-Ray Powder Diffraction Pattern of the 2-propanol solvate

FIG. 9 is an X-Ray Powder Diffraction Pattern of Amorphous Drug Substance

FIG. 10 is a Fourier Transform Infrared Spectrum of the ethanol solvate

FIG. 11 is a Fourier Transform Infrared Spectrum of the 2-propanol solvate

FIG. 12 is a Thermogravimetric Analysis Trace of the ethanol solvate

FIG. 13 is a Thermogravimetric Analysis Trace of the 2-propanol solvate

### DETAILED DESCRIPTION OF THE INVENTION

As used above, and throughout the description of the invention, various terms used herein shall have the generally accepted meanings in the art. More particularly, the following terms, unless otherwise indicated, shall generally be understood to have the following meanings.

“Amorphous” means a solid that it is in a non-crystalline state. Amorphous solids generally possess crystal-like short range molecular arrangement, but no long range order of molecular packing as are found in crystalline solids. The solid state form of a solid, such as the amorphous form of N—[(S)-2(S)-1-azabicyclo[2.2.2]oct-2-yl(phenyl)methyl]-2,6-dichloro-3-(trifluoromethyl)benzamide hydrochloride, may be determined by Polarized Light Microscopy, X-Ray Powder Diffraction (XPRD), Differential Scanning calorimetry (DSC), or other standard techniques known to those of skill in the art.

The amorphous form of N—[(S)-2(S)-1-azabicyclo[2.2.2]oct-2-yl(phenyl)methyl]-2,6-dichloro-3-(trifluoromethyl)benzamide hydrochloride according to the present invention preferably contains less than about 50% by weight, preferably less than 25% by weight, and more preferably less than about 10% by weight of any crystalline forms of the drug substance.

“Compounds of the invention,” as used herein, is meant to describe Form A of N—[(S)-2(S)-1-azabicyclo[2.2.2]oct-2-yl(phenyl)methyl]-2,6-dichloro-3-(trifluoromethyl)benzamide hydrochloride in substantially pure form, Form B of N—[(S)-2(S)-1-azabicyclo[2.2.2]oct-2-yl(phenyl)methyl]-2,6-dichloro-3-(trifluoromethyl)benzamide hydrochloride, N—[(S)-2(S)-1-azabicyclo[2.2.2]oct-2-yl(phenyl)methyl]-2,6-dichloro-3-(trifluoromethyl)benzamide hydrochloride ethanol solvate, N—[(S)-2(S)-1-azabicyclo[2.2.2]oct-2-yl(phenyl)methyl]-2,6-dichloro-3-(trifluoromethyl)benzamide hydrochloride 2-propanol solvate, and amorphous N—[(S)-2(S)-1-azabicyclo[2.2.2]oct-2-yl(phenyl)methyl]-2,6-dichloro-3-(trifluoromethyl)benzamide hydrochloride.

The term “drug substance,” as used herein, refers to N—[(S)-2(S)-1-azabicyclo[2.2.2]oct-2-yl(phenyl)methyl]-2,6-dichloro-3-(trifluoromethyl)benzamide hydrochloride in any form.

“Form A,” as used herein, is meant to describe a crystalline form of N—[(S)-2(S)-1-azabicyclo[2.2.2]oct-2-yl(phenyl)methyl]-2,6-dichloro-3-(trifluoromethyl)benzamide hydrochloride that may be characterized using distinguishing data as described herein. Form A is also synonymously called “polymorph Form A.”

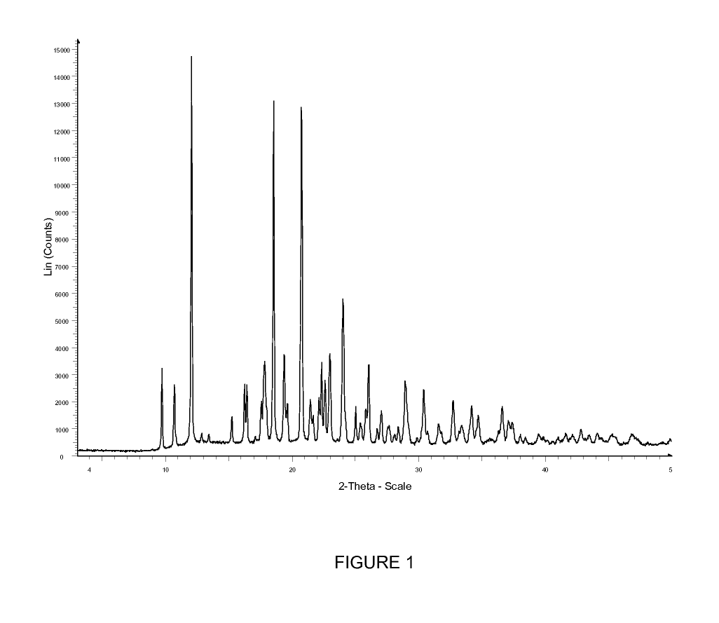

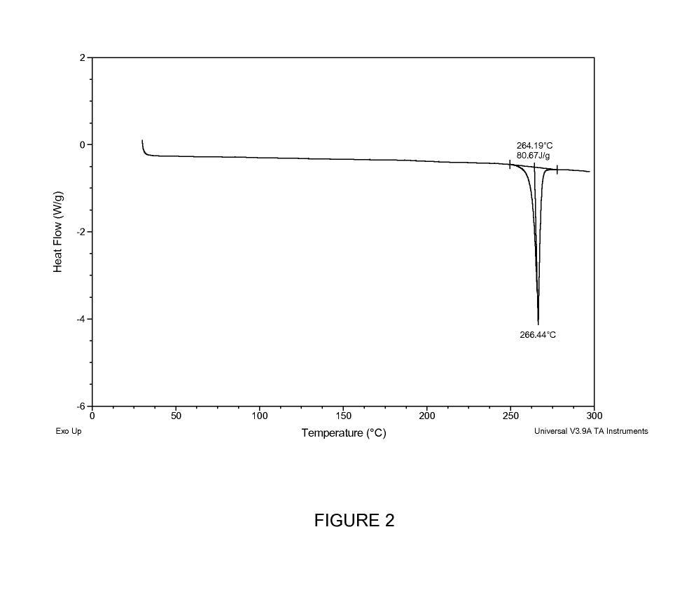

“Form A in substantially pure form,” as used herein, is meant to describe Form A, as defined above, that is substantially free of residual organic solvent contaminants or impurities. By substantially free, it is meant that Form A contains less than 0.5%, and preferably less than 0.1% each of residual solvents or impurities. Exemplary data is found in FIGS. 1, 2, and/or 3.

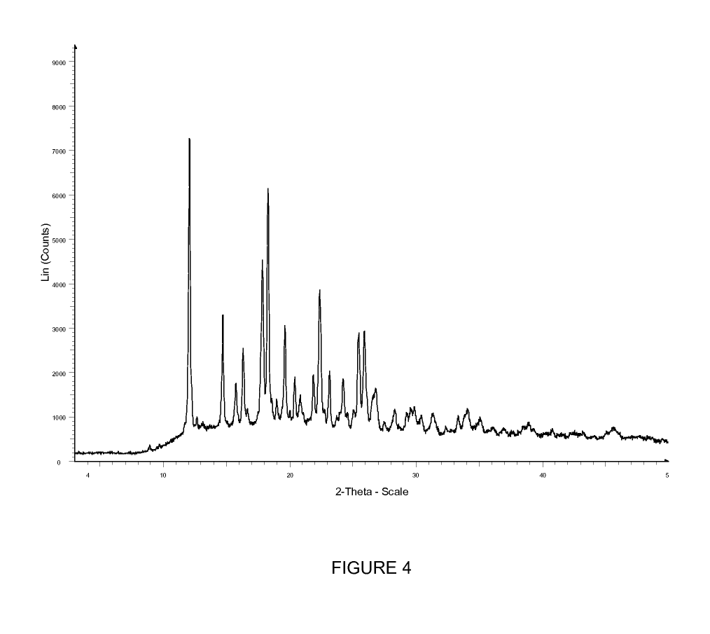

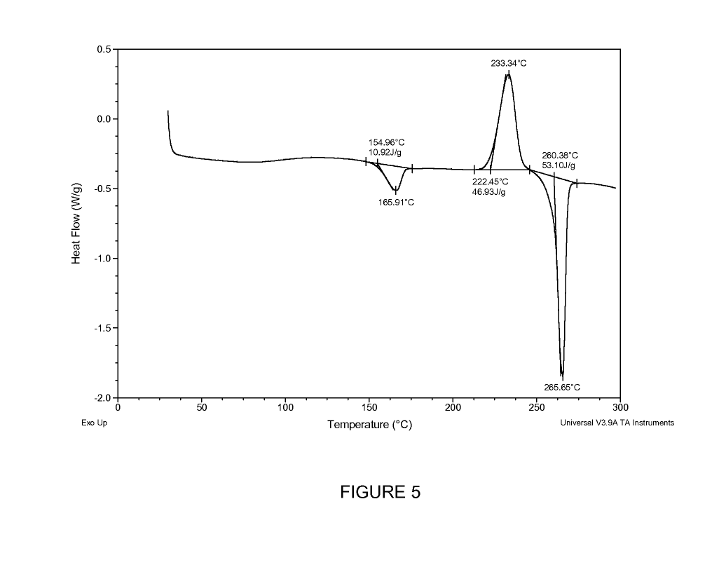

“Form B,” as used herein, is meant to describe a crystalline form of N—[(S)-2(S)-1-azabicyclo[2.2.2]oct-2-yl(phenyl)methyl]-2,6-dichloro-3-(trifluoromethyl)benzamide hydrochloride that may be characterized using distinguishing data as described herein. Exemplary data is found in FIGS. 4, 5, and/or 6. Form B is also synonymously called “polymorph Form B.”

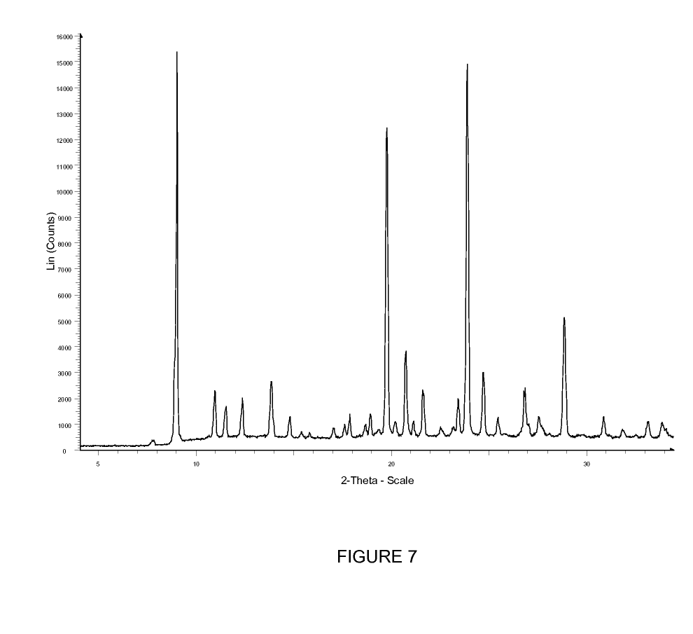

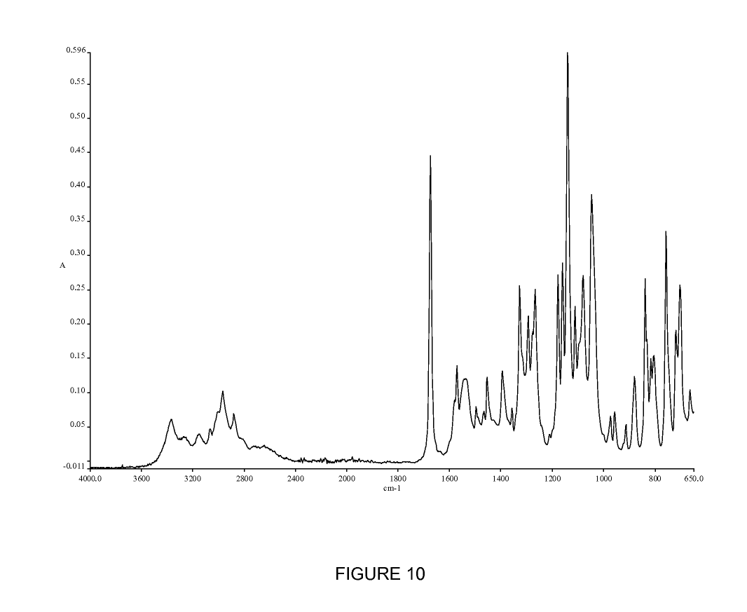

“Ethanol solvate,” as used herein, is meant to describe a crystalline ethanol solvate form of N—[(S)-2(S)-1-azabicyclo[2.2.2]oct-2-yl(phenyl)methyl]-2,6-dichloro-3-(trifluoromethyl)benzamide hydrochloride that may be characterized using distinguishing data as described herein. Exemplary data is found in FIGS. 7, 10 and/or 12. The ethanol solvate is also synonymously called “N—[(S)-2(S)-1-azabicyclo[2.2.2]oct-2-yl(phenyl)methyl]-2,6-dichloro-3-(trifluoromethyl)benzamide hydrochloride ethanol solvate.”

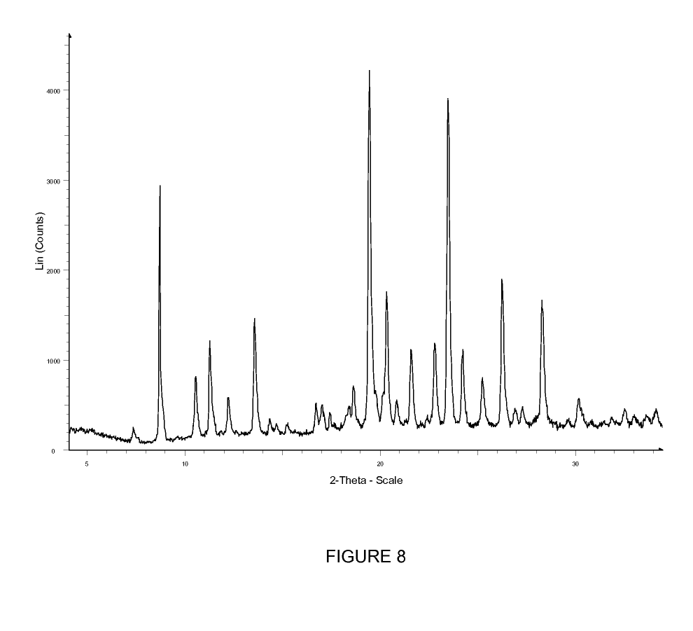

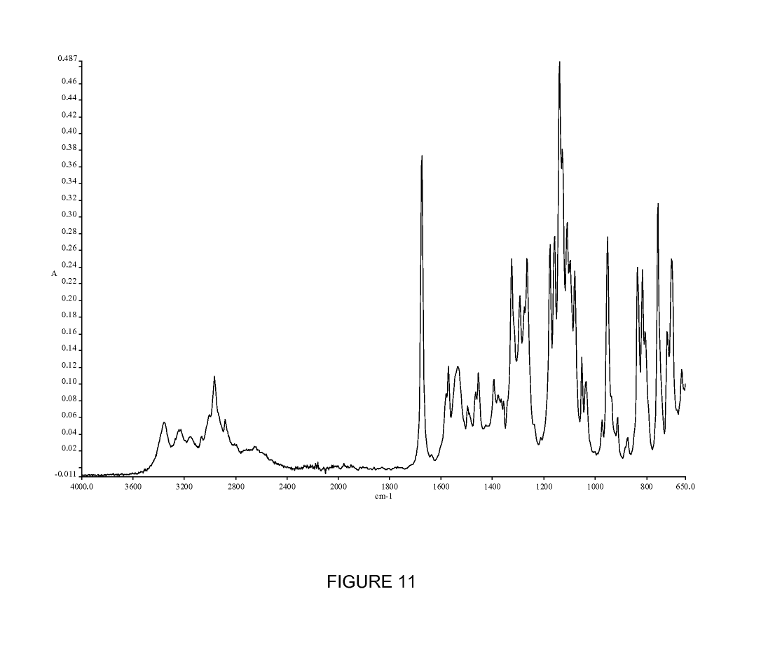

“2-Propanol solvate,” as used herein, is meant to describe a crystalline 2-propanol solvate form of N—[(S)-2(S)-1-azabicyclo[2.2.2]oct-2-yl(phenyl)methyl]-2,6-dichloro-3-(trifluoromethyl)benzamide hydrochloride that may be characterized using distinguishing data as described herein. Exemplary data is found in FIGS. 8, 11, and/or 13. The 2-propanol solvate is also synonymously called “N—[(S)-2(S)-1-azabicyclo[2.2.2]oct-2-yl(phenyl)methyl]-2,6-dichloro-3-(trifluoromethyl)benzamide hydrochloride 2-propanol solvate.”

“Treating” or “treatment” means to alleviate or partially alleviate symptoms, eliminate the causation of the symptoms either on a temporary or permanent basis, or to slow the appearance of symptoms of the named disorder or condition. The compounds and compositions of this invention are useful in treating a pathology in which an inhibitor GLYT-1 provides a therapeutic benefit. For example, the treatment of schizophrenia may include improving cognitive deficits and reducing positive symptoms.

“Patient” includes both human and other mammals.

“Pharmaceutically effective amount” is meant to describe an amount of a compound, composition, medicament or other active ingredient effective in producing the desired therapeutic effect.

It was surprising and unexpectedly discovered that when the free base of N′—(S)-(2S)-1-azabicyclo[2.2.2]oct-2-yl(phenyl)methyl]-2,6-dichloro-3-(trifluoromethyl)benzamide is dissolved in ethanol or 2-propanol with hydrochloric acid, the result is the formation a novel ethanol solvate and 2-propanol solvate compound, respectively. When the ethanol solvate was dried, it forms a new polymorph “Form B.” When the 2-propanol solvate is dried, it forms a new largely amorphous form. Form A prepared from Form B or the amorphous form according to the present invention limits the retention of the undesirable, residual organic solvents. It should be noted that Form B need not be dried completely in order to obtain Form A in substantially pure form. For example, Form B after drying may contain about 5% or less of ethanol.

Form B has lower stability than Form A, but is also highly soluble in water (>200 mg/ml) and will dissolve at very high concentrations. This form readily converts to the desired polymorph A in an aqueous system which is highly stable.

Dissolving any form of N—[(S)-2(S)-1-azabicyclo[2.2.2]oct-2-yl(phenyl)methyl]-2,6-dichloro-3-(trifluoromethyl)benzamide hydrochloride, for example Form A, in ethanol leads to the formation of an ethanol solvate. De-solvation of this form by, for example, drying, leads to the formation of a new material referred to as Form B. Form A, particularly Form A in substantially pure form, may be obtained from Form B by crystallizing from a suitable solvent, such as water.

Dissolving any form of N—[(S)-2(S)-1-azabicyclo[2.2.2]oct-2-yl(phenyl)methyl]-2,6-dichloro-3-(trifluoromethyl)benzamide hydrochloride, for example Form A, in 2-propanol leads to the formation of a 2-propanol solvate. Desolvation of this form by, for example, drying, leads to a highly amorphous sample with low levels of crystallinity. Form A, particularly Form A in substantially pure form, may be obtained from the desolvated 2-propanol solvate by crystallizing from a suitable solvent, such as water.

Form A in substantially pure form can be prepared by the novel procedures of the present invention.

In one aspect, a process for preparing Form A in substantially pure form comprises two steps. In step 1, Form B or the amorphous N—[(S)-2(S)-1-azabicyclo[2.2.2]oct-2-yl(phenyl)methyl]-2,6-dichloro-3-(trifluoromethyl)benzamide hydrochloride is formed from N—[(S)-2(S)-1-azabicyclo[2.2.2]oct-2-yl(phenyl)methyl]-2,6-dichloro-3-(trifluoromethyl)benzamide free base, which is subsequently followed by step 2 in which Form B or the amorphous form is converted to Form A in the presence of water by one of two methods, either seeding and cooling, which leads to a thick slurry with poor flow properties or by seeding and holding at a raised temperature at which polymorph conversion takes place, leading to a more free-flowing slurry. By carefully selecting this raised hold temperature, the particle size distribution of the obtained polymorph Form A may be controlled.

In one aspect, the present invention relates to a process for preparing Form A, said process comprising dissolving N—[(S)-2(S)-1-azabicyclo[2.2.2]oct-2-yl(phenyl)methyl]-2,6-dichloro-3-(trifluoromethyl)benzamide hydrochloride in an alcohol, for example ethanol or 2-propanol, to form an alcohol solvate of N—[(S)-2(S)-1-azabicyclo[2.2.2]oct-2-yl(phenyl)methyl]-2,6-dichloro-3-(trifluoromethyl)benzamide hydrochloride, for example the ethanol or 2-propanol solvate; desolvating the alcohol solvate, to form desolvated N—[(S)-2(S)-1-azabicyclo[2.2.2]oct-2-yl(phenyl)methyl]-2,6-dichloro-3-(trifluoromethyl)benzamide hydrochloride, for example Form B or the amorphous form; and interacting the desolvated N—[(S)-2(S)-1-azabicyclo[2.2.2]oct-2-yl(phenyl)methyl]-2,6-dichloro-3-(trifluoromethyl)benzamide hydrochloride with water to form Form A, and preferably Form A in substantially pure form.

In another aspect, Form A is prepared by dissolving N—[(S)-2(S)-1-azabicyclo[2.2.2]oct-2-yl(phenyl)methyl]-2,6-dichloro-3-(trifluoromethyl)benzamide hydrochloride in an alcohol, for example ethanol or 2-propanol, to form a solution; seeding the solution with Form B to convert the drug substance to Form B; and suspending Form B in water to form Form A, preferably Form A in substantially pure form.

In another aspect, the present invention relates to a process for preparing Form A, said process comprising mixing the free base form of N—[(S)-2(S)-1-azabicyclo[2.2.2]oct-2-yl(phenyl)methyl]-2,6-dichloro-3-(trifluoromethyl)benzamide in an organic solvent, such as ethanol; interacting the free base of N—[(S)-2(S)-1-azabicyclo[2.2.2]oct-2-yl(phenyl)methyl]-2,6-dichloro-3-(trifluoromethyl)benzamide with hydrochloric acid; forming a solvate form of N—[(S)-2(S)-1-azabicyclo[2.2.2]oct-2-yl(phenyl)methyl]-2,6-dichloro-3-(trifluoromethyl)benzamide hydrochloride, for example an ethanol solvate; desolvating the solvate form of N—[(S)-2(S)-1-azabicyclo[2.2.2]oct-2-yl(phenyl)methyl]-2,6-dichloro-3-(trifluoromethyl)benzamide hydrochloride to form Form B of N—[(S)-2(S)-1-azabicyclo[2.2.2]oct-2-yl(phenyl)methyl]-2,6-dichloro-3-(trifluoromethyl)benzamide hydrochloride; and mixing Form B of N—[(S)-2(S)-1-azabicyclo[2.2.2]oct-2-yl(phenyl)methyl]-2,6-dichloro-3-(trifluoromethyl)benzamide hydrochloride with water to form Form A of N—[(S)-2(S)-1-azabicyclo[2.2.2]oct-2-yl(phenyl)methyl]-2,6-dichloro-3-(trifluoromethyl)benzamide hydrochloride. In one aspect of the invention, Form B is dissolved in water in a ratio no greater than 1:4 by weight and, in particular Form B is dissolved in water in a ratio in the range 1:1 to 1:2. One aspect of the invention further comprises the step of drying Form A. In another aspect, the process comprises dissolving Form B of N—[(S)-2(S)-1-azabicyclo[2.2.2]oct-2-yl(phenyl)methyl]-2,6-dichloro-3-(trifluoromethyl)benzamide hydrochloride in water, and allowing Form A to crystallize, forming an aqueous slurry. Form A of N—[(S)-2(S)-1-azabicyclo[2.2.2]oct-2-yl(phenyl)methyl]-2,6-dichloro-3-(trifluoromethyl)benzamide hydrochloride may then be isolated by filtering the aqueous slurry. In a particular aspect, the aqueous slurry of Form A is diluted with water (for example, with a water ratio of about 1:3 to 1:6 by weight) prior to isolating Form A to increase the flowability of the slurry for isolation. In another aspect, the Form A is Form A in substantially pure form.

Another aspect of the invention is a process for preparing Form A, said process comprising mixing the free base form of N—[(S)-2(S)-1-azabicyclo[2.2.2]oct-2-yl(phenyl)methyl]-2,6-dichloro-3-(trifluoromethyl)benzamide in an organic solvent, such as 2-propanol; interacting the free base of N—[(S)-2(S)-1-azabicyclo[2.2.2]oct-2-yl(phenyl)methyl]-2,6-dichloro-3-(trifluoromethyl)benzamide with hydrochloric acid; forming a solvate form of N—[(S)-2(S)-1-azabicyclo[2.2.2]oct-2-yl(phenyl)methyl]-2,6-dichloro-3-(trifluoromethyl)benzamide hydrochloride, for example a 2-propanol solvate; desolvating the solvate form of N—[(S)-2(S)-1-azabicyclo[2.2.2]oct-2-yl(phenyl)methyl]-2,6-dichloro-3-(trifluoromethyl)benzamide hydrochloride to form amorphous N—[(S)-2(S)-1-azabicyclo[2.2.2]oct-2-yl(phenyl)methyl]-2,6-dichloro-3-(trifluoromethyl)benzamide hydrochloride; and mixing amorphous N—[(S)-2(S)-1-azabicyclo[2.2.2]oct-2-yl(phenyl)methyl]-2,6-dichloro-3-(trifluoromethyl)benzamide hydrochloride with a suitable solvent such as water to form Form A of N—[(S)-2(S)-1-azabicyclo[2.2.2]oct-2-yl(phenyl)methyl]-2,6-dichloro-3-(trifluoromethyl)benzamide hydrochloride. One aspect of the invention further comprises the step of drying Form A. In another aspect, the process comprises dissolving the amorphous form of N—[(S)-2(S)-1-azabicyclo[2.2.2]oct-2-yl(phenyl)methyl]-2,6-dichloro-3-(trifluoromethyl)benzamide hydrochloride in water, and allowing Form A to crystallize, forming an aqueous slurry. Form A of N—[(S)-2(S)-1-azabicyclo[2.2.2]oct-2-yl(phenyl)methyl]-2,6-dichloro-3-(trifluoromethyl)benzamide hydrochloride may then be isolated by filtering the aqueous slurry. In one aspect, the Form A is Form A in substantially pure form. In a particular aspect, the aqueous slurry is diluted prior to filtration to increase the flowability of the slurry.

Also provided is a process for preparing Form B of N—[(S)-2(S)-1-azabicyclo[2.2.2]oct-2-yl(phenyl)methyl]-2,6-dichloro-3-(trifluoromethyl)benzamide hydrochloride comprising desolvating N—[(S)-2(S)-1-azabicyclo[2.2.2]oct-2-yl(phenyl)methyl]-2,6-dichloro-3-(trifluoromethyl)benzamide hydrochloride ethanol solvate to form Form B of N—[(S)-2(S)-1-azabicyclo[2.2.2]oct-2-yl(phenyl)methyl]-2,6-dichloro-3-(trifluoromethyl)benzamide hydrochloride.

Another process for preparing Form B of N—[(S)-2(S)-1-azabicyclo[2.2.2]oct-2-yl(phenyl)methyl]-2,6-dichloro-3-(trifluoromethyl)benzamide hydrochloride comprises dissolving N—[(S)-2(S)-1-azabicyclo[2.2.2]oct-2-yl(phenyl)methyl]-2,6-dichloro-3-(trifluoromethyl)benzamide hydrochloride in an alcohol, such as ethanol, and adding seed crystals of Form B to the solution.

A particular process for preparing Form B comprises mixing the free base form of N—[(S)-2(S)-1-azabicyclo[2.2.2]oct-2-yl(phenyl)methyl]-2,6-dichloro-3-(trifluoromethyl)benzamide in an organic solvent, for example an alcohol such as ethanol; interacting the free base of N—[(S)-2(S)-1-azabicyclo[2.2.2]oct-2-yl(phenyl)methyl]-2,6-dichloro-3-(trifluoromethyl)benzamide with hydrochloric acid; forming a solvate form of N—[(S)-2(S)-1-azabicyclo[2.2.2]oct-2-yl(phenyl)methyl]-2,6-dichloro-3-(trifluoromethyl)benzamide hydrochloride, for example an ethanol solvate; and desolvating the solvate form of N—[(S)-2(S)-1-azabicyclo[2.2.2]oct-2-yl(phenyl)methyl]-2,6-dichloro-3-(trifluoromethyl)benzamide hydrochloride to form Form B of N—[(S)-2(S)-1-azabicyclo[2.2.2]oct-2-yl(phenyl)methyl]-2,6-dichloro-3-(trifluoromethyl)benzamide hydrochloride.

Another aspect of the invention is a process for preparing N—[(S)-2(S)-1-azabicyclo[2.2.2]oct-2-yl(phenyl)methyl]-2,6-dichloro-3-(trifluoromethyl)benzamide hydrochloride form B directly from N—[(S)-2(S)-1-azabicyclo[2.2.2]oct-2-yl(phenyl)methyl]-2,6-dichloro-3-(trifluoromethyl)benzamide free base and hydrochloric acid without first isolating the solvated form. This method comprises dissolving N—[(S)-2(S)-1-azabicyclo[2.2.2]oct-2-yl(phenyl)methyl]-2,6-dichloro-3-(trifluoromethyl)benzamide in ethanol; adding hydrochloric acid; seeding the solution with Form B crystals; and isolating Form B.

Another aspect of the invention is a process for preparing N—[(S)-2(S)-1-azabicyclo[2.2.2]oct-2-yl(phenyl)methyl]-2,6-dichloro-3-(trifluoromethyl)benzamide hydrochloride ethanol solvate comprising interacting the free base form of N—[(S)-2(S)-1-azabicyclo[2.2.2]oct-2-yl(phenyl)methyl]-2,6-dichloro-3-(trifluoromethyl)benzamide with hydrochloric acid in ethanol and obtaining the ethanol solvate solid.

In a particular process, the ethanol solvate is prepared by crystallizing N—[(S)-2(S)-1-azabicyclo[2.2.2]oct-2-yl(phenyl)methyl]-2,6-dichloro-3-(trifluoromethyl)benzamide hydrochloride in ethanol.

An aspect of the invention is a process for preparing N—[(S)-2(S)-1-azabicyclo[2.2.2]oct-2-yl(phenyl)methyl]-2,6-dichloro-3-(trifluoromethyl)benzamide hydrochloride 2-propanol solvate comprising interacting the free base form of N—[(S)-2(S)-1-azabicyclo[2.2.2]oct-2-yl(phenyl)methyl]-2,6-dichloro-3-(trifluoromethyl)benzamide with hydrochloric acid in 2-propanol and obtaining the 2-propanol solvate solid.

In a particular process, the 2-propanol solvate is prepared by crystallizing N—[(S)-2(S)-1-azabicyclo[2.2.2]oct-2-yl(phenyl)methyl]-2,6-dichloro-3-(trifluoromethyl)benzamide hydrochloride in 2-propanol.

Another aspect of the invention is a process for preparing the amorphous form of N—[(S)-2(S)-1-azabicyclo[2.2.2]oct-2-yl(phenyl)methyl]-2,6-dichloro-3-(trifluoromethyl)benzamide hydrochloride comprising desolvating N—[(S)-2(S)-1-azabicyclo[2.2.2]oct-2-yl(phenyl)methyl]-2,6-dichloro-3-(trifluoromethyl)benzamide hydrochloride 2-propanol solvate to form amorphous N—[(S)-2(S)-1-azabicyclo[2.2.2]oct-2-yl(phenyl)methyl]-2,6-dichloro-3-(trifluoromethyl)benzamide hydrochloride.

A particular aspect of the invention is a process for preparing the amorphous form of N—[(S)-2(S)-1-azabicyclo[2.2.2]oct-2-yl(phenyl)methyl]-2,6-dichloro-3-(trifluoromethyl)benzamide hydrochloride comprising mixing the free base form of N—[(S)-2(S)-1-azabicyclo[2.2.2]oct-2-yl(phenyl)methyl]-2,6-dichloro-3-(trifluoromethyl)benzamide in an organic solvent, for example an alcohol such as 2-propanol; interacting the free base of N—[(S)-2(S)-1-azabicyclo[2.2.2]oct-2-yl(phenyl)methyl]-2,6-dichloro-3-(trifluoromethyl)benzamide with hydrochloric acid; forming a solvate form of N—[(S)-2(S)-1-azabicyclo[2.2.2]oct-2-yl(phenyl)methyl]-2,6-dichloro-3-(trifluoromethyl)benzamide hydrochloride, for example the 2-propanol solvate; and desolvating the solvate form of N—[(S)-2(S)-1-azabicyclo[2.2.2]oct-2-yl(phenyl)methyl]-2,6-dichloro-3-(trifluoromethyl)benzamide hydrochloride to form amorphous N—[(S)-2(S)-1-azabicyclo[2.2.2]oct-2-yl(phenyl)methyl]-2,6-dichloro-3-(trifluoromethyl)benzamide hydrochloride.

The present invention provides pharmaceutical compositions comprising one or more compounds of the invention in combination with one or more pharmaceutically acceptable carrier agents, bulking agents, solvents, diluents and other excipients. In one aspect, the pharmaceutical compositions comprise Form A in substantially pure form

Another aspect of the invention is a pharmaceutical composition prepared by formulating one or more compounds of the invention, for example, Form A in substantially pure form, with one or more pharmaceutically acceptable carrier agents, bulking agents, solvents, diluents and other excipients.

The present invention also provides a process for preparing a pharmaceutical composition comprising formulating one or more compounds of the invention with one or more pharmaceutically acceptable carrier agents, bulking agents, solvents, diluents and other excipients. One aspect of the invention is a process for preparing a pharmaceutical composition comprising formulating Form A in substantially pure form with one or more pharmaceutically acceptable carrier agents, bulking agents, solvents, diluents and other excipients.

The compounds and compositions of the present invention are useful in the treatment of pathologies in which an inhibitor of GLYT-1 provides a therapeutic benefit, for example, neurological disorders such as schizophrenia.

Accordingly, an aspect of the present invention is a method of treating a pathology in which an inhibitor of GLYT-1 provides a therapeutic benefit.

Another aspect of the invention is a method of treating pathologies selected from the group consisting of dementia, psychoses, such as schizophrenia (deficient form and productive form), neuro-degenerative disorders, acute or chronic extra-pyramidal symptoms induced by neuroleptics, anxiety, panic attacks, phobias, obsessive-compulsive disorders, depression, including psychotic depression, and the like; said method comprising administering to a patient in need thereof a pharmaceutically effective amount of a compound of the invention.

Another aspect of the invention is the use of a compound of the invention for the treatment of a disease selected from the group consisting of schizophrenia (deficient form and productive form), neuro-degenerative disorders, acute or chronic extra-pyramidal symptoms induced by neuroleptics, anxiety, panic attacks, phobias, obsessive-compulsive disorders, depression, including psychotic depression, and the like.

The following examples detailed below are provided to more specifically describe and to better teach how to make and practice the claimed invention. The examples are provided for illustrative purposes only. Therefore, the examples should not be interpreted as limiting the spirit and scope of the invention as later recited by the claims that follow. Suitable N—[(S)-2(S)-azabicyclo[2.2.2]oct-2-yl(phenyl)methyl]-2,6-dichloro-3-trifluoromethyl)benzamide starting material for the herein described procedures includes, but is not limited to, N—[(S)-2(S)-azabicyclo[2.2.2]oct-2-yl(phenyl)methyl]-2,6-dichloro-3-trifluoromethyl)benzamide prepared according to the procedures described in U.S. Pat. No. 7,288,656. In some instances, such as for commercial scale synthesis, it may be advantageous to seed with the desired crystalline form during the preparation of such form.

### Example 1

### Preparation of Ethanol Solvate and Form B

N—[(S)-2(S)-1-azabicyclo[2.2.2]oct-2-yl(phenyl)methyl]-2,6-dichloro-3-(trifluoromethyl)benzamide hydrochloride (7.01 g) was dissolved in 35 mL of ethanol (grade 3C 200prf) at 55° C. The resultant solution was left to cool down to room temperature (20° C.) over a period of 2 hours whereupon a thick slurry was obtained. After filtering the slurry, the wet cake corresponded to the ethanol solvate. Upon drying overnight in a vacuum oven (60° C., −25 mm Hg) a total of 5.68 g of dried solid, corresponding to form B, was obtained (overall yield=81%).

### Example 2

### Preparation of Form B from Free Base

N—[(S)-2(S)-azabicyclo[2.2.2]oct-2-yl(phenyl)methyl]-2,6-dichloro-3-trifluoromethyl)benzamide (21.9 g) was dissolved at room temperature (RT) in ethanol (67 ml, EtOH; grade 3C 200 prf) and mixed until a clear solution was formed, which took 35 minutes and comprised approximately 83 mL of solution. The resultant solution was polish-filtered, and the reactor was rinsed with a further amount of ethanol (16 ml) which was combined with the filtrate leading to a total volume of 99 ml. 33 mL of the filtered solution was transferred to a Multimax 50 mL reactor cell. The solution contained 7.3 g of the free base compound per se.

The solution was then stirred and heated to 55° C. and to this was quickly added concentrated aqueous HCl (2.18 mL 12.1 N), which resulted in a clear solution after 20 minutes of stirring. The temperature was then lowered to 50° C. at a rate of 1° C./min. Once this temperature limit was reached, the solution was seeded with approximately 0.1% crystals of Form B of N—[(S)-2(S)-1-azabicyclo[2.2.2]oct-2-yl(phenyl)methyl]-2,6-dichloro-3-(trifluoromethyl)benzamide hydrochloride. The temperature was held constant for 30 minutes at which point the slurry was cooled to −5° C. at 0.5° C./min, and then filtered under a vacuum. The filtration was fast and resulted in a wet cake net weight of 9.62 g which was then dried at 60° C. under a vacuum (100 mbar) for 2 hours. The resulting cake which weighed 7.03 g, was then dried for another 5 days at 50° C. (70 mbar) to provide 7.00 g (87%) of the desired solid.

### Example 3

### Preparation of 2-Propanol Solvate, Amorphous Form, and Form A in Substantially Pure Form

2.53 g of Form A of N—[(S)-2(S)-1-azabicyclo[2.2.2]oct-2-yl(phenyl)methyl]-2,6-dichloro-3-(trifluoromethyl)benzamide hydrochloride were added to a flask along with 2-propanol (27.9 ml). The contents were heated to 70° C. and stirred to facilitate dissolution. The contents were then left to cool to room temperature and precipitation was noted at approximately 30° C. The contents were filtered, and the solid was determined to be the 2-propanol solvate. After drying the solids overnight in a vacuum oven at 85° C., 400 mm Hg, the solid was determined to be essentially amorphous. Taking 1.97 g of this amorphous material and loading into 5.13 mL of water held at 55° C. initially led to dissolution followed by clouding. Upon cooling to room temperature, a poorly flowable solid was recovered which flowed better after an additional amount of water (0.92 ml) were added. Upon filtration and drying in a vacuum oven overnight at 85° C., 400 mm Hg the final solid was identified as form A.

### Example 4

### Preparation of Form B to Form A

2 g of the HCl salt of polymorph Form B was placed into a reaction vessel. In a second reaction vessel, deionized water was heated to 55° C. 4 mL of the heated water was added to the vessel containing the polymorph Form B solids. This was the stirred at a speed of 250 rpm. The slurry was left to stir at 55° C. for 10 minutes, which resulted in a clear solution. The polymorph Form A of N—[(S)-2(S)-1-azabicyclo[2.2.2]oct-2-yl(phenyl)methyl]-2,6-dichloro-3-(trifluoromethyl)benzamide hydrochloride (8.3 mg of seed crystals), was dispersed into 250 μl of water and added to the solution. The seeds did not dissolve. The turbidity of the solution increased significantly leading to a thick, white slurry. The slurry was cooled at a rate of 0.5° C./min down to 1.0° C. At a temperature of 48° C., the slurry became thick and non-flowing. The cooling was continued at a rate of 0.5° C./min until the temperature of the reaction vessel reached 18.6° C.

At 18.6° C., the slurry, still very thick and non-flowing, was filtered. Since the slurry could not be poured from the vial, a spatula had to be used to remove the solids. The vial was washed with 3 mL of water and the wash was filtered. The filter cake, the Buchner funnel, and the vial were placed into the oven at 60° C. and <100 mbar pressure and dried overnight. Yield: 85.8%

### Example 5

### Modified Procedure for the Conversion of Form B to Form A

A 100 mL reactor was loaded with 60 g of demineralized water. The water was stirred at 500 rpm and heated to 45° C. at which point 10 g of Form B powder was added. Form B is highly soluble in water and dissolved readily. The temperature was held at 45° C. for 3 hours and the resultant free flowing slurry was cooled to 1° C. at 0.2° C./min where it was held for 8.5 hours. The resulting free-flowing milky suspension was unloaded, filtered and dried at 70° C. under 200 mm Hg vacuum with a light nitrogen flow for 12 hours. The product is confirmed as Form A with a final yield of 88%.

### Comparative Examples

Comparative Example Lots A through E were prepared by cooling or antisolvent crystallization.

Table I below lists the amount of solvent found in drug substance prepared by this method. The residual solvents are entrapped in the crystals making their removal impossible by conventional drying techniques.

The Examples of Lots F-I were prepared according to the processes of the present invention. As shown in Table 1 below, the amounts of residual solvents in these novel polymorph forms (Lots F-I), are significantly lower than those found in the final products of the processes of the Comparative Procedures that do not result in the formation of these novel polymorphic forms.

TABLE 1

| Lot | Total residual organic solvents, ppm | Comments |
|---|---|---|
| A | 13,000 (EtOH), 7,000 (MTBE) | Comparative Procedure |
| B | 500 (MTBE), 400 (2-Me—THF) | Comparative Procedure |
| C | 12,000 (EtOH), 7.000 (MTBE) | Comparative Procedure |
| D | 9,000 (EtOH), 36,000 (EtOAc) | Comparative Procedure |
| E | 5,100 (1-butanol) | Comparative Procedure |
| F | <100 (EtOH) | Process of the invention |
| | | (Form A) |
| G | <100 (EtOH) | Process of the invention |
| | | (Form B) |
| H | <100 (EtOH) | Process of the invention |
| | | (Form A) |
| I | 14 (EtOH) | Process of the invention |
| | | (Form A) |

The compounds of the invention are analyzed by the following analytical methods.

### Experimentals

X-Ray Power Diffractometry (XRPD)

X-ray powder diffractometry is performed on a Bruker D8 Advance diffractometer using the parafocusing Bragg-Brentano (theta-two-theta)-type geometry. The compound of the invention, as a powder, is deposited on a single-crystal silicon wafer, cut according to the (510) crystallographic orientation. Copper K-alpha radiation (1.54056 angstroms), emitted from a copper anode tube (45 kV/40 mA) is used as the x-ray source with a divergence slit of 0.5 mm. A LynxEye detector is used to collect diffracted beams. The diffraction pattern is obtained using the following conditions: at least 3.0 to 30.0 degree scan in angle 2-theta, 0.5 second count time per step, 0.02 degree step size, under ambient conditions of pressure, temperature, and relative humidity.

FIG. 1 is an XRPD pattern of Form A in substantially pure form. Table 2 sets forth the characteristic peak locations, d-spacings and relative intensities for Form A.

TABLE 2
Characteristic XRPD Peak locations and Relative Intensities of the Form A

| | Calculated | |
|---|---|---|
| Measured Angle | Spacing | Relative |
| Degrees 2θ +/− | d value | Intensity |
| 0.2° 2θ | (Angstroms) | (%) |
| 9.7 | 9.14 | 35 |
| 10.7 | 8.30 | 18 |
| 12.0 | 7.35 | 100 |
| 18.5 | 4.79 | 84 |
| 20.7 | 4.29 | 62 |
| 24.0 | 3.71 | 53 |

In particular, the peaks at 9.7±0.2 and 10.7±0.2 in 2θ are characteristic of Form A.

FIG. 4 is an XRPD pattern of Form B. Table 3 sets forth the characteristic peak locations, d-spacings and relative intensities for Form B.

TABLE 3
Characteristic XRPD Peak locations and Relative Intensities of the Form B

| | Calculated | |
|---|---|---|
| Measured Angle | Spacing | Relative |
| Degrees 2θ +/− | d value | Intensity |
| 0.2° 2θ | (Angstroms) | (%) |
| 12.0 | 7.34 | 100 |
| 14.7 | 6.04 | 45 |
| 18.2 | 4.85 | 84 |
| 19.6 | 4.53 | 42 |
| 22.4 | 3.98 | 53 |

In particular, the peaks the peaks at 14.7±0.2 and 18.2±0.2 in 2θ are characteristic of Form B.

FIG. 7 is an XRPD pattern of the ethanol solvate. Table 4 sets forth the characteristic peak locations, d-spacings and relative intensities for the ethanol solvate.

TABLE 4
Characteristic XRPD Peak locations and Relative Intensities of the
ethanol solvate

| | Calculated | |
|---|---|---|
| Measured Angle | Spacing | Relative |
| Degrees 2θ +/− | d value | Intensity |
| 0.2° 2θ | (Angstroms) | (%) |
| 7.7 | 11.47 | 8 |
| 8.9 | 9.90 | 48 |
| 11.5 | 7.72 | 15 |
| 13.8 | 6.42 | 32 |
| 19.7 | 4.50 | 100 |
| 23.8 | 3.73 | 99 |

In particular, the peaks the peaks at 8.9, 11.5 and 13.8 in 28 are characteristic of the ethanol solvate.

FIG. 8 is an XRPD pattern of the 2-propanol solvate. Table 5 sets forth the characteristic peak locations, d-spacings and relative intensities for the 2-propanol solvate.

TABLE 5
Characteristic XRPD Peak locations and Relative Intensities of the
2-propanol solvate

| | Calculated | |
|---|---|---|
| Measured Angle | Spacing | Relative |
| Degrees 2θ +/− | d value | Intensity |
| 0.2° 2θ | (Angstroms) | (%) |
| 7.3 | 12.04 | 20 |
| 8.7 | 10.19 | 47 |
| 11.3 | 7.84 | 53 |
| 13.6 | 6.52 | 55 |
| 19.4 | 4.57 | 100 |
| 23.5 | 3.79 | 75 |

In particular, the peaks the peaks at 8.7, 11.3 and 13.6 in 28 are characteristic of the 2-propanol solvate.

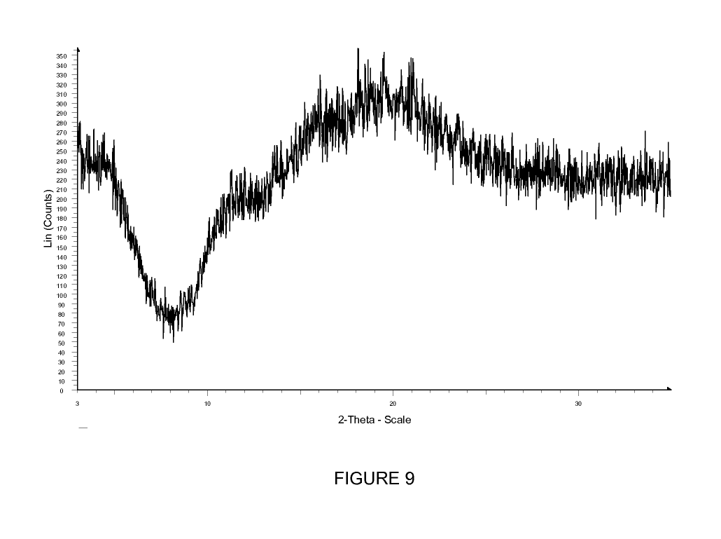

FIG. 9 is an X-Ray Powder Diffraction Pattern of the amorphous drug substance prepared by desolvating 2-propanol solvate. The lack of well-defined peaks in the X-ray powder diffraction pattern demonstrates the amorphous nature of the material.

A person skilled in the art will recognize that the peak locations could be slightly affected by differences in sample height. The peak locations described herein are thus subject to a variation of plus or minus (+/−) 0.2 degrees 2-theta. The relative intensities may change depending on crystallite size and morphology.

Differential Scanning Calorimetry

Differential scanning calorimetry was carried out using a TA Instruments Q200 instrument. Samples were heated in hermetically sealed pans at 10° C. per minute from 30° C. up to 300° C.

The Differential Scanning calorimetry trace of Form A as shown in FIG. 2 illustrates a clean melt around 265° C.

The Differential Scanning calorimetry trace of Form B depicted in FIG. 5 illustrates the melt with an onset at approximately 155° C. followed by re-crystallization and subsequent melting behavior.

Fourier Transform Infrared Spectroscopy (FTIR)

Fourier Transform IR spectra were obtained using a Perkin Elmer Spectrum One Spectrometer with a uATR accessory. The samples were scanned from 4000 cm⁻¹ to 650 cm⁻¹, 4 times and a spectral resolution of 4 cm⁻¹ was used.

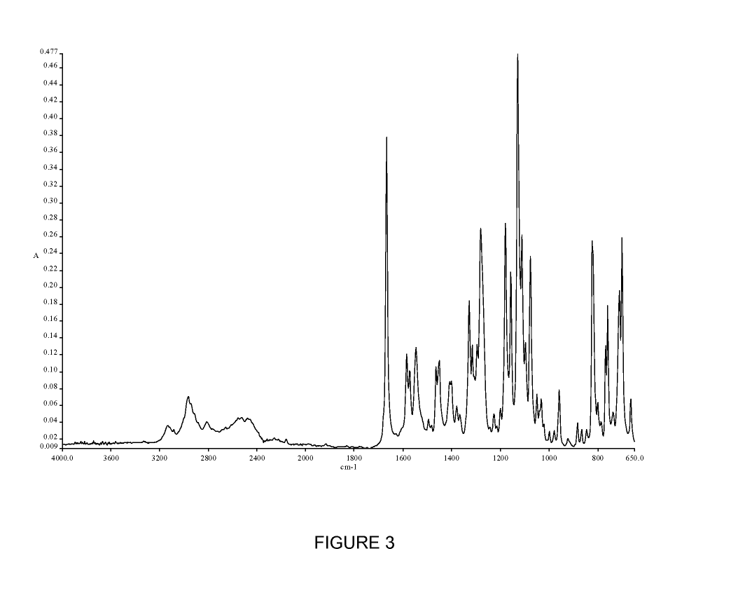

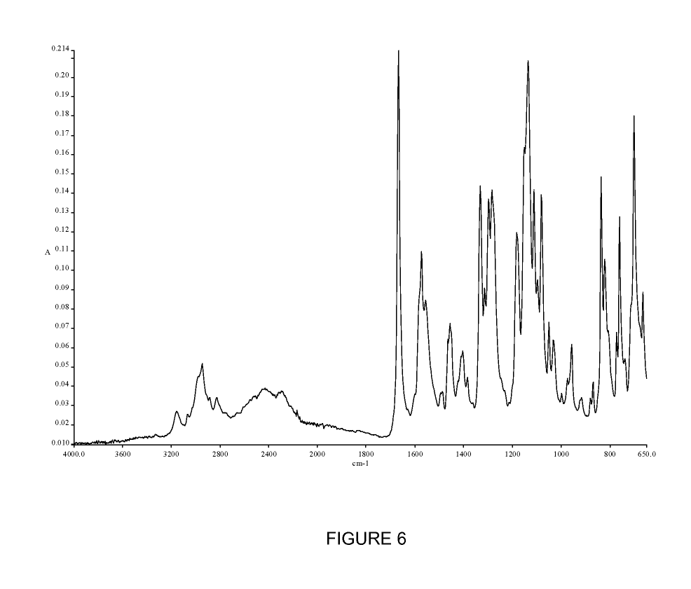

The FTIR spectra of the compounds prepared essentially according to Example 2 (FIG. 6) and Example 4 (FIG. 3) are consistent with the chemical structure of N—[(S)-2(S)-1-azabicyclo[2.2.2]oct-2-yl(phenyl)methyl]-2,6-dichloro-3-(trifluoromethyl)benzamide hydrochloride.

FIG. 3 is an FTIR of form A. Characteristic wavenumbers for Form A of N—[(S)-2(S)-1-azabicyclo[2.2.2]oct-2-yl(phenyl)methyl]-2,6-dichloro-3-(trifluoromethyl)benzamide hydrochloride include, but are not limited to 1547, 1158 and 1130 cm⁻¹.

FIG. 6 is an FTIR of form B. Characteristic wavenumbers for Form B of N—[(S)-2(S)-1-azabicyclo[2.2.2]oct-2-yl(phenyl)methyl]-2,6-dichloro-3-(trifluoromethyl)benzamide hydrochloride include, but are not limited to 1136 and 837 cm⁻¹.

FIG. 10 is an FTIR of the ethanol solvate. Characteristic wavenumbers for the ethanol solvate of N—[(S)-2(S)-1-azabicyclo[2.2.2]oct-2-yl(phenyl)methyl]-2,6-dichloro-3-(trifluoromethyl)benzamide hydrochloride include, but are not limited to 1047 and 958 cm⁻¹.

FIG. 11 is an FTIR of the 2-propanol solvate. Characteristic wavenumbers for the 2-propanol solvate of N—[(S)-2(S)-1-azabicyclo[2.2.2]oct-2-yl(phenyl)methyl]-2,6-dichloro-3-(trifluoromethyl)benzamide hydrochloride include, but are not limited to 1053, 1037 and 953 cm⁻¹.

Thermogravimetric Analysis (TGA)

Thermogravimetric analysis was carried out using a TA Instruments Q500 Instrument. Samples were heated in open pans from ambient up to 300° C. at a rate of 20° C. per minute.

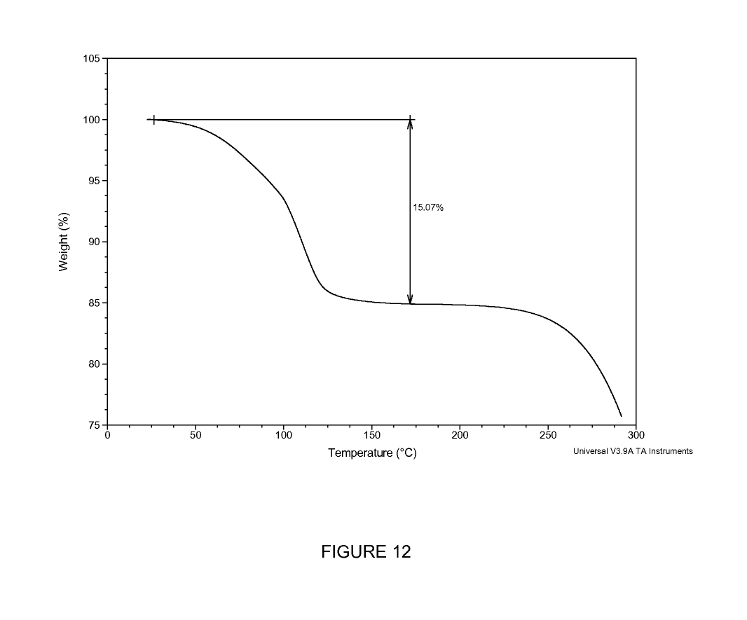

FIG. 12 is a TGA of N—[(S)-2(S)-1-azabicyclo[2.2.2]oct-2-yl(phenyl)methyl]-2,6-dichloro-3-(trifluoromethyl)benzamide hydrochloride ethanol solvate. Loss of weight prior to melting is indicative of desolvation. The weight loss measured approximates to a disolvate.

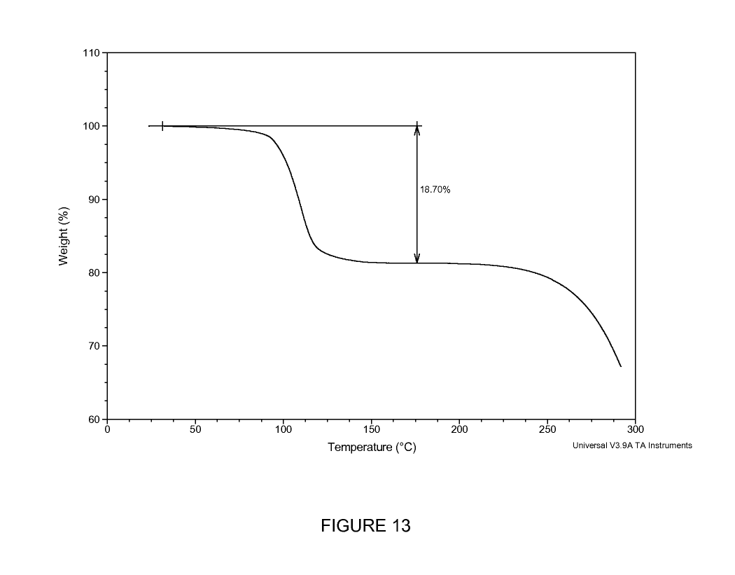

FIG. 13 is a TGA of N—[(S)-2(S)-1-azabicyclo[2.2.2]oct-2-yl(phenyl)methyl]-2,6-dichloro-3-(trifluoromethyl)benzamide hydrochloride 2-propanol solvate. Loss of weight prior to melting is indicative of desolvation. The weight loss measured approximates to a disolvate.

What is claimed is:

1. A crystalline or amorphous form of a compound of formula I

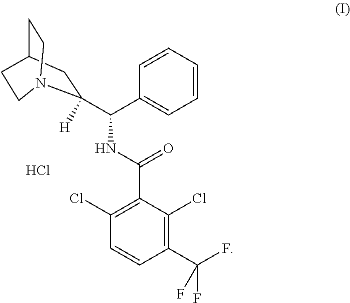

2. The crystalline form of N—[(S)-2(S)-1-azabicyclo[2.2.2]oct-2-yl(phenyl)methyl]-2,6-dichloro-3-trifluoromethyl)benzamide hydrochloride according to claim 1 which is designated as Form A in substantially pure form.

3. The crystalline form according to claim 2 having a melting point of about 262-266° C.

4. The crystalline form according to claim 2 having an X-ray Diffraction Pattern comprising peaks at about 9.7±0.2 and 10.7±0.2 in 2θ.

5. The crystalline form according to claim 4, wherein the an X-ray powder diffraction pattern further comprises peaks at about 18.5±0.2, 20.7±0.2 and 24.0±0.2° in 2θ.

6. The crystalline according to claim 2 having an FT-IR spectrum comprising a peak at about 1130 cm⁻¹.

7. The crystalline form of N—[(S)-2(S)-1-azabicyclo[2.2.2]oct-2-yl(phenyl)methyl]-2,6-dichloro-3-trifluoromethyl)benzamide hydrochloride according to claim 1 which is designated as Form B.

8. The crystalline form according to claim 7 having a melting point of about 150-170° C. followed by a re-crystallization and subsequent melt at about 260-265° C.

9. The crystalline form according to claim 7 having an X-ray Diffraction Pattern comprising peaks at about 14.7±0.2, 18.2±0.2, in degrees 2θ.

10. The crystalline form according to claim 9 having an X-ray Diffraction Pattern further comprising the peaks at about 19.6±0.2 and 22.4±0.2 in degrees 2θ.

11. The crystalline form according to claim 7 having an FT-IR spectrum comprising a peak at about 837 cm⁻¹.

12. The crystalline form of N—[(S)-2(S)-1-azabicyclo[2.2.2]oct-2-yl(phenyl)methyl]-2,6-dichloro-3-trifluoromethyl)benzamide hydrochloride according to claim 1 which is an alcohol solvate.

13. The crystalline form of N—[(S)-2(S)-1-azabicyclo[2.2.2]oct-2-yl(phenyl)methyl]-2,6-dichloro-3-trifluoromethyl)benzamide hydrochloride according to claim 1 which is an ethanol solvate.

14. The crystalline form according to claim 13 having an X-ray Diffraction Pattern comprising peaks at about 8.9±0.2, 11.5±0.2 and 13.8±0.2 in degrees 2θ.

15. The crystalline form according to claim 13 having an FT-IR spectrum comprising a peak at about 1047 cm⁻¹.

16. The crystalline form of N—[(S)-2(S)-1-azabicyclo[2.2.2]oct-2-yl(phenyl)methyl]-2,6-dichloro-3-trifluoromethyl)benzamide hydrochloride according to claim 1 which is a 2-propanol solvate.

17. The crystalline form according to claim 16 having an X-ray Diffraction Pattern comprising peaks at about 8.7±0.2, 11.3±0.2 and 13.6±0.2 in degrees 2θ in degrees 2θ.

18. The crystalline form according to claim 16 having an FT-IR spectrum comprising a peak at about 953 cm⁻¹.

19. An amorphous form of N—[(S)-2(S)-1-azabicyclo[2.2.2]oct-2-yl(phenyl)methyl]-2,6-dichloro-3-trifluoromethyl)benzamide hydrochloride according to claim 1.

20. A process for the preparation of N—[(S)-2(S)-1-azabicyclo[2.2.2]oct-2-yl(phenyl)methyl]-2,6-dichloro-3-(trifluoromethyl)benzamide hydrochloride comprising mixing N—[(S)-2(S)-1-azabicyclo[2.2.2]oct-2-yl(phenyl)methyl]-2,6-dichloro-3-(trifluoromethyl)benzamide in an organic solvent; interacting N—[(S)-2(S)-1-azabicyclo[2.2.2]oct-2-yl(phenyl)methyl]-2,6-dichloro-3-(trifluoromethyl)benzamide with hydrochloric acid to form a solvate of the hydrochloric salt of N—[(S)-2(S)-1-azabicyclo[2.2.2]oct-2-yl(phenyl)methyl]-2,6-dichloro-3-(trifluoromethyl)benzamide; desolvating said solvate to form a Form B of N—[(S)-2(S)-1-azabicyclo[2.2.2]oct-2-yl(phenyl)methyl]-2,6-dichloro-3-(trifluoromethyl)benzamide hydrochloride or an amorphous form of N—[(S)-2(S)-1-azabicyclo[2.2.2]oct-2-yl(phenyl)methyl]-2,6-dichloro-3-(trifluoromethyl)benzamide hydrochloride; mixing said Form B or amorphous form with water to form Form A of N—[(S)-2(S)-1-azabicyclo[2.2.2]oct-2-yl(phenyl)methyl]-2,6-dichloro-3-(trifluoromethyl)benzamide hydrochloride.

21. The process according to claim 20 further comprising drying Form A of N—[(S)-2(S)-1-azabicyclo[2.2.2]oct-2-yl(phenyl)methyl]-2,6-dichloro-3-(trifluoromethyl)benzamide hydrochloride.

22. The process as recited in claim 20 wherein the organic solvent is selected from ethanol and 2-propanol.

23. A process for the preparation of N—[(S)-2(S)-1-azabicyclo[2.2.2]oct-2-yl(phenyl)methyl]-2,6-dichloro-3-(trifluoromethyl)benzamide hydrochloride comprising mixing N—[(S)-2(S)-1-azabicyclo[2.2.2]oct-2-yl(phenyl)methyl]-2,6-dichloro-3-(trifluoromethyl)benzamide hydrochloric in an organic solvent to form a solvate; desolvating said solvate to form a Form B of N—[(S)-2(S)-1-azabicyclo[2.2.2]oct-2-yl(phenyl)methyl]-2,6-dichloro-3-(trifluoromethyl)benzamide hydrochloride or an amorphous form of N—[(S)-2(S)-1-azabicyclo[2.2.2]oct-2-yl(phenyl)methyl]-2,6-dichloro-3-(trifluoromethyl)benzamide hydrochloride; mixing said Form B or amorphous form with water to form Form A of N—[(S)-2(S)-1-azabicyclo[2.2.2]oct-2-yl(phenyl)methyl]-2,6-dichloro-3-(trifluoromethyl)benzamide hydrochloride.

24. A method for the treatment of a neurological disorder selected from the group consisting of schizophrenia, neuro-degenerative disorders, acute or chronic extra-pyramidal symptoms induced by neuroleptics, anxiety, panic attacks, phobias, obsessive-compulsive disorders, and depression, comprising administering to a patient in need thereof a pharmaceutically effective amount of a compound according to claim 1.

25. The method according to claim 24 wherein the compound is Form A of N—[(S)-2(S)-1-azabicyclo[2.2.2]oct-2-yl(phenyl)methyl]-2,6-dichloro-3-(trifluoromethyl)benzamide hydrochloride in substantially pure form.

26. A pharmaceutical composition comprising a compound according to claim 1 in combination with one or more pharmaceutically acceptable carrier agents, bulking agents, solvents, diluents and other excipients.

27. The pharmaceutical composition according to claim 26 wherein the compound is Form A of N—[(S)-2(S)-1-azabicyclo[2.2.2]oct-2-yl(phenyl)methyl]-2,6-dichloro-3-(trifluoromethyl)benzamide hydrochloride in substantially pure form.
